(19) United States
(12) Patent Application Publication
(10) Pub. No.: US 2012/0226261 A1
(43) Pub. Date: Sep. 6, 2012

(54) Intramammary Injection Device

(75) Inventors: Michael A. Barnett, Nashville, TN (US); John R. Dodam, Columbia, MO (US); John R. Middleton, Columbia, MO (US); Miranda N. Shaw, Warsaw, IN (US); Xandra M. Sifuentes, Overland Park, KS (US)

(73) Assignee: THE CURATORS OF THE UNIVERSITY OF MISSOURI, Columbia, MO (US)

(21) Appl. No.: 13/368,471

(22) Filed: Feb. 8, 2012

Related U.S. Application Data
(60) Provisional application No. 61/462,921, filed on Feb. 8, 2011.
(60) Provisional application No. 61/518,734, filed on May 11, 2011.

(51) Int. Cl. A61M 5/315

(52) U.S. Cl. 604/514; 604/232; 604/199

(57) ABSTRACT

In various embodiments, the present disclosure provides an injection system for injecting a fluidic treatment substance into an orifice of an animal. In various implementations the system comprises a treatment cartridge having a hollow body that is structured and operable to retain a treatment substance within the hollow body. The system further comprises a plunger assembly structured and operable to removably retain the treatment cartridge and controllably dispense the treatment substance from the treatment cartridge. In various implementations the injection system can additionally include an injection site cleaning adaptor that is removably connectable to the treatment cartridge and is structured and operable to clean a site in which the treatment substance is to be injected.

### CROSS-REFERENCE TO RELATED APPLICATIONS

This application claims the benefit of U.S. Provisional Application No. 61/462,921, filed on Feb. 8, 2011 and U.S. Provisional Application No. 61/518,734, filed on May 11, 2011. The disclosures of the above applications are incorporated herein by reference in its entirety.

### FIELD

The present teachings relate to an injection device for delivery of fluidic products to a subject.

### BACKGROUND

The statements in this section merely provide background information related to the present disclosure and can not constitute prior art.

Mastitis, an inflammation of a cow's udder usually caused by a bacterial intramammary infection, is a common infectious disease of dairy cattle (about 33% of dairy cows develop mastitis). Moreover, treatment of mastitis is very costly (over $2 billion per year to US dairy industry). Additionally, mastitis is the most common reason dairy cows are treated with antibiotics and is second, only to reproductive failure, as the reason for culling a dairy cow. According to the 2007 data by USDA, about 16.4% of dairy cows (over 1.5 million) in the US were treated with antibiotics for mastitis in 2007; about 90% of dairy operations (over 62,000) used intramammary antibiotics at dry-off; and about 80% of dairy operations (over 50,000) that used intramammary antibiotics at dry-off used intramammary antibiotic treatments on all cows at the operation regardless of necessity for treatment. In addition to, or as an alternative to, antibiotics, sealants are often injected into the teats to prevent intramammary infections.

The current treatments, e.g., antibiotic, sealants or other products, are delivered into the teat and gland cisterns via manual injection through the teat orifice, as illustrated in FIG. 8. The common procedure for administering such products requires an administrator (such as a clinician, a farmer, or other farm personnel) first to clean/disinfect the teat end, discard the cleaning pad, and then inject the product using a preloaded syringe. It is common in some operations that an administrator disinfects multiple teats prior to making an injection. The current operation is time-consuming, labor-intensive, and prone to contamination. Furthermore, the repetitive hand and arm movements (such as the pushing motion by the thumb of an administrator) can be an occupational hazard that induces repetitive motion disorder.

### SUMMARY

In various embodiments, the present disclosure provides an injection system for injecting a fluidic treatment substance into an orifice of an animal. In various implementations, the system comprises a treatment cartridge having a hollow body that is structured and operable to retain a treatment substance within the hollow body. The system further comprises a plunger assembly structured and operable to removably retain the treatment cartridge and controllably dispense the treatment substance from the treatment cartridge.

In various other embodiments, the present disclosure provides a treatment cartridge for use with a plunger assembly for injecting a fluidic treatment substance into an orifice of an animal. The cartridge generally includes a hollow body, an injection nozzle extending from the hollow body, and a push plate slideably disposed within the hollow body. The push plate is structured and operable to move within hollow body, in response to a force applied by a plunger assembly. This movement is operable to force a treatment substance disposed within the hollow body through the nozzle to expel the treatment substance from the hollow body. The cartridge additionally includes one or more cartridge retention structures that are structured and operable to mate with the one or more dock retention structures of the plunger assembly to removably retain the treatment cartridge within the plunger assembly.

In various other embodiments, the present disclosure provides a method for injecting a fluidic treatment substance into an orifice of an animal, wherein the method includes removably retaining a treatment cartridge, comprising an injection nozzle extending from a hollow body and having a treatment substance disposed within the hollow body, within a plunger assembly comprising a handle. The plunger assembly includes a plunger rod slideably disposed within the handle, and a lever bar operatively connected to the plunger rod. The method additionally includes moving the lever bar orthogonally toward the handle to advance the plunger rod within the handle so that the plunger rod will interact with the treatment cartridge to expel the treatment substance through the injection nozzle, thereby dispensing the treatment substance from the treatment cartridge.

Further areas of applicability of the present teachings will become apparent from the description provided herein. It should be understood that the description and specific examples are intended for purposes of illustration only and are not intended to limit the scope of the present teachings.

### DRAWINGS

The drawings described herein are for illustration purposes only and are not intended to limit the scope of the present teachings in any way.

FIG. 1 is an isometric view of an injection system, in accordance with various embodiments of the present disclosure.

FIG. 2(a) is a schematic illustration of an injection site cleaning adaptor of the injection system shown in FIG. 1, in accordance with various embodiments of the present disclosure.

FIG. 2(b) is a schematic illustration of the injection site cleaning adaptor of the injection system shown in FIG. 1, in accordance with various other embodiments of the present disclosure.

FIG. 3(a) is a schematic illustration of the injection site cleaning adaptor of the injection system shown in FIG. 1, in accordance with yet various other embodiments of the present disclosure.

FIG. 3(b) is a schematic illustration of the injection site cleaning adaptor of the injection system shown in FIG. 1, in accordance with still various other embodiments of the present disclosure.

FIG. 4(a) is an isometric view of the injection site cleaning adaptor of the injection system shown in FIG. 1 having a conical shaped body and an injection nozzle cap, in accordance with various other embodiments of the present disclosure.

FIG. 4(b) is a cross-sectional view of the injection site cleaning adaptor shown in FIG. 4(a), in accordance with various other embodiments of the present disclosure.

FIG. 5 is an isometric view of an exemplary plunger assembly of the injection system shown in FIG. 1 configured in a disengaged position, in accordance with various embodiments of the present disclosure.

FIG. 6 is an isometric view of the plunger assembly shown in FIG. 5 configured in an engaged position, in accordance with various embodiments of the present disclosure.

FIG. 7 is an isometric view of an exemplary treatment cartridge of the injection system shown in FIG. 1 that is removably engageable with the plunger assembly shown in FIG. 5 and mateable with the injection site cleaning adaptor shown in FIGS. 1-4(b), in accordance with various embodiments of the present disclosure.

FIG. 8 is an illustration of the injection system shown in FIG. 1 in use, in accordance with various embodiments of the present disclosure.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements.

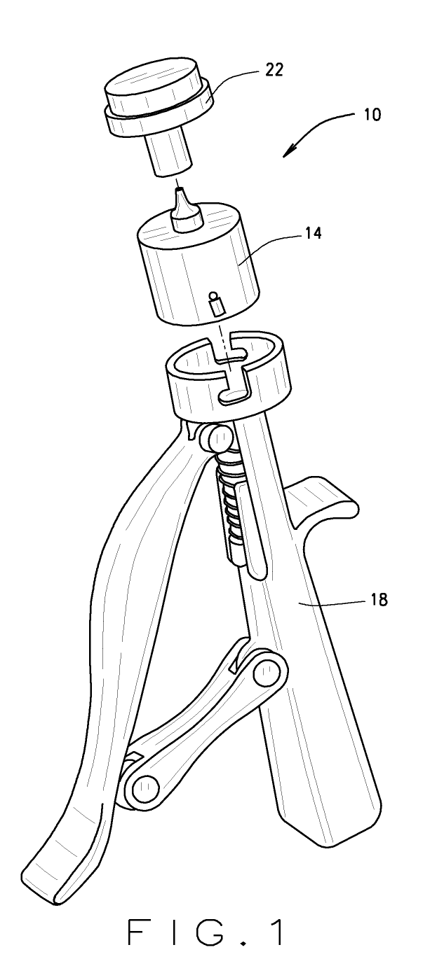

Referring to FIG. 1, in accordance with various embodiments, the present disclosure provides an injection system 10 structured and operable to inject a fluidic treatment substance, e.g., an antibiotic, sealant or other liquid, gel or hydrogel, into an orifice of an animal, e.g., a teat of a cow or goat. Generally, the injection system 10 includes a pre-filled treatment cartridge 14 structured and operable to retain and controllably dispense the treatment substance, and an ergonomic squeeze-motion plunger assembly 18 structured and operable to removably retain the treatment cartridge 14 and control dispensing of the treatment substance from the treatment cartridge 14. Additionally, in various embodiments, the injection system 10 can include an injection site cleaning adaptor 22 that is removably connectable to the treatment cartridge 14 and is structured and operable to clean or sterilize the site in which the treatment substance is to be injected, e.g., the tip and orifice of the teat of a cow.

In various exemplary applications, the injection system 10 can be employed for aseptic delivery of intramammary products into the teat and gland cisterns of a dairy animal. Such intramammary products can include, but are limited to, bismuth subnitrate, paraffin, chlorhexidine and other anti-infective agents, penicillins, cephalosporins, tetracyclines, and lincosamides. When used with the cleaning adaptor 22, the injection system 10 permits aseptic preparation of the injection site, e.g., the teat end, and delivery of the treatment substance in series by removably pre-attaching the cleaning adaptor 22 onto the treatment cartridge 14, or the end of a suitably adapted syringe. It is envisioned that the injection system 10 can be utilized to inject treatment substance into areas of an animal other than the teats.

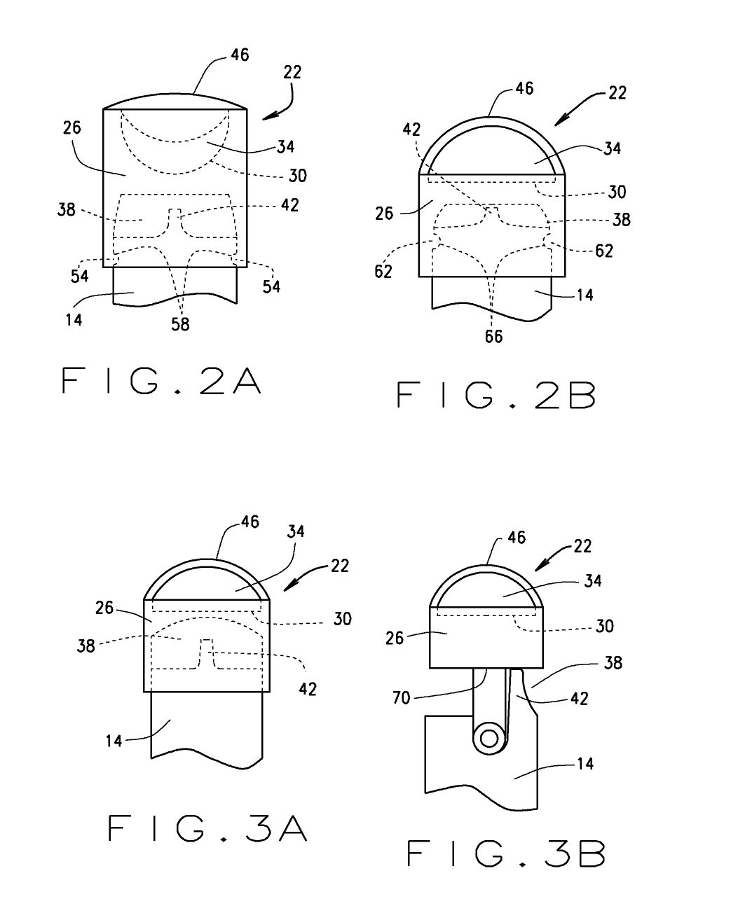

Referring to FIGS. 2(a) through 4(b), in various implementations, the injection site cleaning adaptor 22 comprises a body 26 that includes a sponge/pad basin 30 for retaining a cleaning sponge or pad 34 pre-soaked with a desired antibacterial or antimicrobial solution. The cleaning adaptor body 26 additionally includes receiving reservoir 38 structured to receive and accommodate an injection nozzle 42 of the treatment cartridge 14, or a syringe, or other injection device. Particularly, the cleaning adaptor 22 is structured and operable to be removably attached to the treatment cartridge 14, or any suitably mateable syringe, over the injection nozzle 42 of the treatment cartridge 14 or suitably mateable syringe.

The cleaning pad basin 30 of the cleaning adaptor 22 can have any shape, contour or profile suitable for retaining the cleaning pad 34, which in turn can have any desired shape, contour or profile. For example, as shown in FIG. 2(a), in various implementations, the pad basin 30 can be concave to retain a cleaning pad 34 that is similarly concave to accommodate the shape and size of a teat of a dairy cow. Or, in other implementations, the basin 30 can have a flat bottom structured to receive a cleaning pad 34 having a flat base and a concave top for accommodating the shape and size of a teat of a dairy cow.

In various other embodiments, as illustrated in FIG. 2(b), the basin 30 can have a flat bottom structured to receive a cleaning pad 34 having a flat base and a convex top. Accordingly, the shape, contour or profile of the cleaning adaptor basin 30 and cleaning pad 34 can be compatible with the shape of the area to be disinfected.

Furthermore, in various embodiments, the cleaning adaptor 22 can include a removable seal 46 disposed over the distal end of the adaptor body 26 such that the seal 46 encloses the cleaning pad 34, thereby reducing or preventing dehydration of the cleaning pad 34 and protecting the cleaning pad 34 from debris and/or contaminants, thereby preserving the integrity of the cleaning pad 34.

As described above, the cleaning adaptor 22 is removably connectable to the treatment cartridge 14 or suitably adapted syringe. More specifically, the cleaning adaptor 22 is removably connectable to the treatment cartridge 14, or suitably adapted syringe, such that the injection site can be cleaned or sterilized using the cleaning pad 34 while cleaning adaptor 22 is attached to the cartridge 14, or syringe. In various embodiments, the cleaning adaptor 22 is connectable to the treatment cartridge 14, or syringe, such that the injection nozzle 42 of the treatment cartridge 14, or syringe, is disposed within the cartridge receiving reservoir 38. Therefore, the cleaning adaptor 22 covers the injection nozzle 42, thereby protecting the injection nozzle 42 from contamination and providing a sterile environment for the injection nozzle 42. Once the injection site has been cleansed, the cleaning adaptor 22 can be removed from the treatment cartridge 14, or syringe, thereby exposing the injection nozzle 42 such that the treatment substance retained therein can be dispensed through the injection nozzle 42, as described below.

The cleaning adaptor 22 and treatment cartridge 14, or syringe, can have any suitable structure that provides for the cleaning adaptor 22 to be removably connectable to the treatment cartridge 14, or syringe. For example, as illustrated in FIG. 2(a), in various embodiments, a proximal end of a wall forming the receiving reservoir 38 can be structured to include a raised lip 54 and the treatment cartridge 14 is structured to include a lip receiving channel 58 structured to receive and mate with the raised lip 54. Accordingly, the cleaning adaptor 22 is retained on the treatment cartridge 14, or syringe, via mating of the raised lip 54 and lip receiving channel 58, while the cleaning pad 34 is utilized to aseptically prepare the injection site. Subsequently, after the aseptic preparation, the cleaning adaptor 22 can be easily snapped off (i.e., removed from) the treatment cartridge 14, or syringe, to expose the injection nozzle 42, via a force sufficient to displace the raised lip 54 from the lip receiving channel 58.

Or, in various other embodiments, as illustrated in FIG. 2(b), the wall forming the receiving reservoir 38 can be structured to include at least one protuberance 62, e.g., a single raised circumferential ridge or a plurality of nodules, formed along the interior of the wall. Correspondingly, the treatment cartridge 14 can be structured to include one or more receptors 66 structured to receive and mate with the one or more protuberances 62. Accordingly, the cleaning adaptor 22 is retained on the treatment cartridge 14, or syringe, via mating of the protuberance(s) 62 and receptor(s) 66, while the cleaning pad 34 is utilized to aseptically prepare the injection site. Subsequently, after the aseptic preparation, the cleaning adaptor 22 can be easily snapped off (i.e., removed from) the treatment cartridge 14, or syringe, to expose the injection nozzle 42, via a force sufficient to displace the protuberance(s) 62 from the receptor(s) 66.

Although the removable connectivity of the cleaning adaptor 22 and the treatment cartridge 14 has been described above having the raised lip 54 and protuberance 62 as part of the cleaning adaptor 22 and the respective receiving channel 58 and receptor 66 as part of the treatment cartridge 14, it is envisioned that, alternatively, the treatment cartridge 14 can include the raised lip 54 or protuberance 62 and the cleaning adaptor 22 can include the respective receiving channel 58 or receptor 66.

In yet other embodiments, as illustrated in FIG. 3(a), the cleaning adaptor 22 can have a simple frictional fit with the treatment cartridge 14. That is the inside diameter of the adaptor receiving reservoir 38 can have substantially the same dimensions as the outside diameter of the treatment cartridge 14. Accordingly, the cleaning adaptor 22 is retained on the treatment cartridge 14, or syringe, via the frictional fit between the inside of the adaptor receiving reservoir wall and outside of the treatment cartridge 14, while the cleaning pad 34 is utilized to aseptically prepare the injection site. Subsequently, after the aseptic preparation, the cleaning adaptor 22 can be easily removed from the treatment cartridge 14, or syringe, to expose the injection nozzle 42, via a force sufficient to overcome the friction between the inside of the adaptor receiving reservoir wall and outside of the treatment cartridge 14.

In still other embodiments, as shown in FIG. 3(b), the cleaning adaptor 22 can be structured to include a pair of pivot connectors, or hinges, 70, that are structured to pivotally connect with the exterior sides of the treatment cartridge 14, or syringe. Accordingly, the cleaning adaptor 22 is retained and positioned over the injection nozzle 42 while the cleaning pad 34 is utilized to aseptically prepare the injection site. Subsequently, after the aseptic preparation, the cleaning adaptor 22 can be easily rotated away from the injection nozzle 42, thereby exposing the injection nozzle 42 for use.

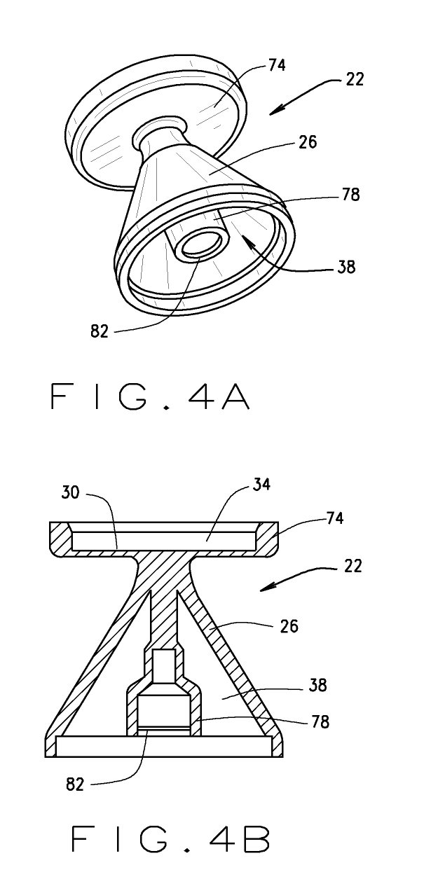

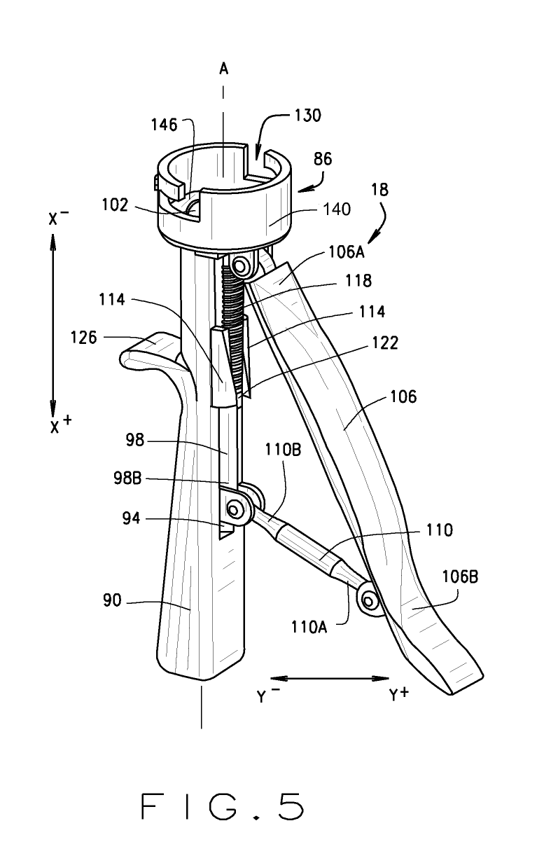

Referring now to FIGS. 4(a) and (b), in various embodiments the cleaning adaptor 22 can be structured to have a conical shaped body 26 and include a cleaning pad pedestal 74 extending from the vertex end of the conical body 26. The cleaning pad basin 30 is formed within the pedestal 74. The conical shape of the cleaning adaptor 22 provides easy handling and removal of the cleaning adaptor 22 after aseptic preparation of the injection site, as described above. In such embodiments, the cleaning adaptor 22 can additionally include an injection nozzle cap 78 disposed with the receiving reservoir 38. The injection nozzle cap 78 is structured to fit over the injection nozzle 42 (shown in FIGS. 2A-3B) of the respective treatment cartridge 14, or syringe, to provide further protection of the injection nozzle 42 from debris and contaminants. More specifically, in various implementations, the injection nozzle cap 78 can include a rim-seal 82, e.g., a gasket or O-ring, that provides a snug or tight fit of the injection nozzle cap 78 on the injection nozzle 42 when the cleaning adaptor is connected to the respective treatment cartridge 14, or syringe. Thus, the injection nozzle cap 78 can provide a substantially air-tight seal around the respective injection nozzle 42.

Referring now to FIGS. 1, 5 and 6, the ergonomically designed plunger assembly 18 is structured and operable to employ a squeezing motion, instead of the thumb-pushing motion of conventional syringes, to dispense the treatment substance contained in the treatment cartridge 14. Moreover, since the treatment cartridge 14 is removably retainable within the plunger assembly 18, the plunger assembly 18 can be reused for multiple treatment cartridges 14, simply by removing, or disengaging, a first used treatment cartridge 14 from the plunger assembly 18 and installing, or engaging, a second unused treatment cartridge 14, as described further below.

In various embodiments, the plunger assembly 18 can include a cartridge docking platform 86 that is structured and operable to removably engage the treatment cartridge 14. The plunger assembly 18 additionally comprises a stationary handle 90 that includes a recessed channel 94 in which a plunger rod 98 is slidingly disposed. The plunger rod 98 includes a plunger cap 102 disposed at a distal end 98A. More specifically, the distal end 98A of the plunger rod 98 extends through an aperture (not shown) in a base of the docking platform 86 such that the plunger cap 102 is disposed within the interior of the docking platform 86.

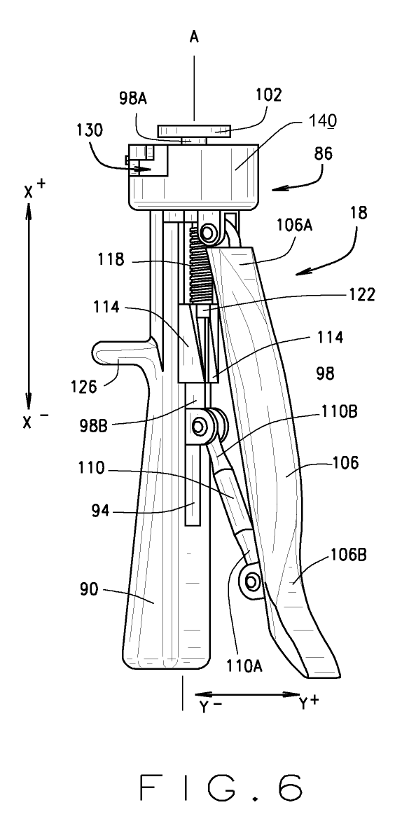

The plunger assembly 18 further includes a lever bar 106 that is pivotally connected to the handle 90 and/or a base of the docking platform 86 at a distal end 106A, and is pivotally connected at proximal end 106B to a proximal end 98B of the plunger rod 98, via a connecting rod 110. Particularly, the connecting rod 110 is pivotally connected at a first end 110A to the proximal end 106B of the lever bar 106 and pivotally connected at a second end 110B to the proximal end 98B of the plunger rod 98. Still more particularly, the connecting rod 110 is pivotally connected to the plunger rod 98 and lever bar 106 such that movement (e.g., squeezing) of the lever bar 106 in a Y⁻ direction from a disengaged position (shown in FIG. 5) toward the stationary handle 90 (as shown in FIG. 6) will cause the plunger rod 98 to move longitudinally in the X⁺ direction within the channel 94. Note that the X⁺ and X⁻ directions are generally parallel or coaxial with an axis A of the plunger assembly 18, while the Y⁺ and Y⁻ directions are generally orthogonal to an axis A of the plunger assembly 18. Consequently, the longitudinal movement of the plunger rod 98 in the X⁺ direction will cause the plunger cap 102 to likewise move in the X⁺ direction within the docking platform 86 (as shown in FIG. 6).

In various embodiments, the plunger assembly 18 can further include a pair of plunger rod guide retainers 114 and a lever return biasing device 118. The plunger rod guide retainers 114 are disposed on, or integrally formed with, the stationary handle 90 along the sides of the channel 94 and are structured and operable to secure and ensure the sliding of the plunger rod 98 within the channel 94. The biasing device 118 is structured and operable to bias movement of the plunger rod 98 in the X⁻ direction, and hence, bias movement of the lever bar 106 in the Y⁺ direction away from the stationary handle 90 and toward the disengaged position (shown in FIG. 5). Particularly, the biasing device 118 is operable to assist in retraction of the plunger rod 98 after the treatment substance has been expelled from the treatment cartridge 14 so that the plunger assembly 18 can be reused with subsequent treatment cartridges 14, as described further below.

The biasing device 118 can be any device suitable to bias the plunger rod 98 in the X⁻ direction, and hence bias the lever bar 106 in the Y⁺ direction toward the disengaged position (shown in FIG. 5), e.g., a coil spring, leaf spring, compressible sleeve/bushing or any other suitable biasing device. For example, in various embodiments, the biasing device 118 can be a coil spring disposed over the plunger rod 98 such that a first end of the spring abuts the bottom of the docking platform 86 and an opposing second end abuts a stop 122 dispose on, or integrally formed with, the plunger rod 98. In such embodiments, the outside diameter of the stop 122 is sized to be slightly less than the inner diameter of the lumen forming the channel 94. Alternatively, the lumen forming the channel 94 can include the stop 122 such that the second end of the spring abuts the stop formed within the channel 94.

In various other embodiments, the plunger assembly 18 can include a hand buttress 126 extending from the stationary handle 90 for assisting a user in maintaining a secure grip on the stationary handle 90 during use of the plunger assembly 18.

Referring now to FIGS. 5, 6 and 7, as described above, the cartridge docking platform 86 is structured and operable to removably engage the treatment cartridge 14. Importantly, the docking platform 86 includes one or more dock retention structures 130 that is/are structured and operable to securely seat, align and removably retain the treatment cartridge 14 within the docking platform 86 throughout use of the injection system 10. More specifically, the dock retention structure 130 is structured and operable to cooperatively and/or interlockingly mate with one or more cartridge retention structures 134 included as part of the treatment cartridge 14. Thus, to seat, align and removably secure the treatment cartridge 14 within the docking platform 86, the user cooperatively and/or interlockingly engages the cartridge retention structure(s) 134 with the dock retention structure 130. The dock and cartridge retention structures 130 and 134 can be any interlocking structures suitable for securely seating, aligning and removably retaining the treatment cartridge 14 within the docking platform 86.

For example, in various embodiments, the cartridge retention structure(s) 134 can comprise two or more retention pins or posts extending from the exterior of a sidewall 136 of a body 138 of the treatment cartridge 14, and the dock retention structure 130 can comprise a corresponding two or more substantially ‘L’ shaped receiving slots formed in a sidewall 140 of the docking platform 86. Accordingly, in such embodiments, to securely seat, align and removably retain the treatment cartridge 14 within the docking platform 86 the user inserts each retention pin 134 into a vertical leg of a corresponding one of the receiving slots 130 until the retention pins 134 contact a bottom of the vertical leg. Subsequently, the user twists, turns, or rotates the treatment cartridge 14 such that the retention pins 134 advance into horizontal legs of the receiving slots 130. Importantly, in such embodiments, the treatment cartridge 14 is structured such that the retention pins 134 extend from the body 138 at a location that causes a top surface of the retention pins 134 to frictionally contact a top edge of the receiving slot's horizontal legs, thereby exerting a slight force in the X⁻ direction on treatment cartridge 14. This slight force in the X⁻ direction firmly engages a bottom edge 142 of the treatment cartridge body 138 against a floor 146 of the docking platform 86, thereby securely seating, aligning and removably retaining the treatment cartridge 14 within the docking platform 86. Subsequently, in such embodiments, to remove a treatment cartridge 14 from the docking platform 86, the user merely twists, turns, or rotates the treatment cartridge 14 in the opposite direction until the retention pins 134 align with the vertical legs of the receiving slots 130, whereafter the treatment cartridge 14 can be removed from the docking platform 86.

Alternatively, the dock and cartridge retention structures 130 and 134 can be any suitable cooperative and/or interlocking structures such as cooperative threads, whereby the treatment cartridge 14 can be screwed, or threaded, into the docking platform 86, or any other cooperative and/or interlocking structures operable to securely seat, align and removably retain the treatment cartridge 14 within the docking platform 86.

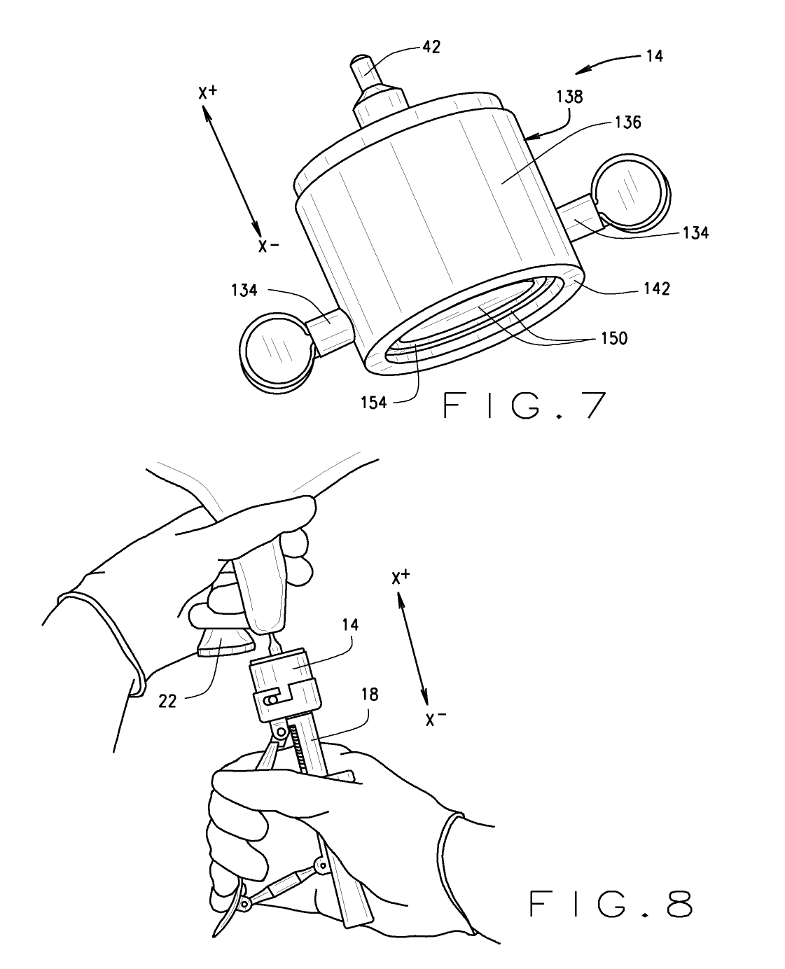

Further referring to FIGS. 6 and 7, as described above, the treatment cartridge 14 includes the hollow cartridge body 138 and the injection nozzle 42 extending from a top of the hollow body 138. Additionally, the treatment cartridge 14 includes a push plate 150 that is slideably disposed within an interior of the cartridge body 138. The selected treatment substance is predisposed within the cartridge hollow body 138. To dispense the treatment substance through the cartridge injection nozzle 42 the treatment cartridge 14 is securely engaged within the docking platform 86, via the mating dock and cartridge retention structures 130 and 134, as described above. As also described above, securely engaging the treatment cartridge 14 with the docking platform 86, via the mating dock and cartridge retention structures 130 and 134, aligns the treatment cartridge 14 within the docking platform 86. More specifically, securely engaging the treatment cartridge 14 with the docking platform 86, via the mating dock and cartridge retention structures 130 and 134, coaxially aligns the plunger cap 102 of the plunger assembly 18 with the push plate 150. Therefore, the plunger cap 102 is centered with the push plate 150 and no portion of the plunger cap is adjacent or aligns with any portion of the bottom edge 142 of the cartridge body 138.

In various embodiments, the plunger cap 102 can be sized to have a diameter that is slightly smaller than the diameter of the push plate 150 to further insure that no portion of the plunger cap 102 is adjacent or aligns with any portion of the bottom edge 142 of the cartridge body 138.

Additionally, as illustrated in FIG. 7, in various embodiments, the treatment cartridge 14 can be structured such that the push plate 150 is slightly recessed within the cartridge body 138. Furthermore, in such embodiments, the plunger assembly 18 can be structured such that the plunger cap 102 extends slightly above the floor 146 of the docking platform 86 when the lever bar 106 is in the disengaged position. Accordingly, when the treatment cartridge 14 is engaged with the docking platform 86 at least a portion of the plunger cap 102 will be disposed within the recess formed between the push plate 150 and the cartridge body bottom edge 142, thereby aiding in the alignment of the plunger cap 102 with the push plate 150.

Once the treatment cartridge 14 is securely engaged within the docking platform 86, the treatment substance can be dispensed from treatment cartridge 14, via the injection nozzle 42, by moving, i.e., squeezing, the plunger assembly lever bar 106 in the Y⁻ direction toward the stationary handle 90. As described above, moving/squeezing the lever bar 106 in the Y⁻ direction toward the stationary handle advances the plunger rod 98, and thus, the plunger cap 102, in the X⁺ direction. Consequently, the plunger cap 102 drives the treatment cartridge push plate 150 in the X⁺ direction forcing the treatment substance disposed within the cartridge body 138 to be expelled, or dispensed, from the treatment cartridge 14 through the injection nozzle 42.

The push plate 150 is sized to have a friction, or interference fit, with the cartridge body sidewall 136 such that a substantially liquid-tight seal is provided between the perimeter edge of the push plate 150 and an interior side of the cartridge body sidewall 136. Alternatively, in various implementations, the push plate 150 can include a seal (not shown), e.g., a gasket or O-ring, disposed within the perimeter edge to provide the substantially liquid-tight seal. The substantially liquid-tight seal prevents or significantly inhibits the treatment substance disposed within the hollow body 138 from leaking between push plate 150 and the cartridge body sidewall 136 as the push plate 150 is advanced in the X⁺ direction, via the force applied by the plunger rod 98 and plunger cap 102, to dispense the treatment substance from the injection nozzle 42.

In various embodiments, push plate 150 can include a debris reservoir 154 for displacing debris and other substances, that may collect on the plunger cap 102 during repeated use of the plunger assembly 18. Particularly, any debris/substances that collects on the plunger cap 102 during use of the plunger assembly 18 can be displaced, i.e., pushed, from the plunger cap 102 into the debris reservoir 154 as the plunger cap 102 contacts/engages the push plate 150 when the treatment cartridge 14 is engaged within docking platform 86 and/or when the plunger rod 98 is advanced driving the push plate 150 in the X⁺ direction. Displacing debris/substances that collect on the plunger cap 102 will help prevent such debris/substances from fouling of the interface between the plunger cap 102 and the push plate 150 and improve the reliability and repeatability of the movement of the plunger cap 102 and push plate 150 within the cartridge body 138 to reliably dispense the treatment substance from a plurality of treatment cartridges 14.

Referring now to FIGS. 1, 5, 6, 7 and 8, as described above (with reference to FIG. 1), in various embodiments, the injection system 10 includes the injection site cleaning adaptor 22, the treatment cartridge 14 and the plunger assembly 18, wherein the treatment cartridge 14 is removably engageable with plunger assembly 18 and the cleaning adaptor 22 is removably connectable to the treatment cartridge 14. As exemplarily illustrated in FIG. 8, in various embodiments, the injection system 10 can be utilized to aseptically prepare the teat of a dairy cow and inject an antibiotic and/or sealant into the teat. More specifically, during an aseptic preparation and injection process, an administrator cleans the dairy cow's teat orifice utilizing the cleaning pad 34 (pre-soaked with a desired antibacterial or antimicrobial solution) of the cleaning adaptor 22 while the cleaning adaptor 22 is removably connected to the treatment cartridge 14. After the teat has been cleansed, the administrator easily removes the cleaning adaptor 22 from the treatment cartridge 14, thereby exposing the injection nozzle 42 of the treatment cartridge 14. The administrator can then insert the injection nozzle 42 into the streak canal. Once the injection nozzle 42 is inserted into the streak canal, the administrator can move/squeeze the lever bar 106 in the Y⁻ direction toward the stationary handle 90, thereby advancing the plunger rod and cap 98 and 102 in the X⁺ direction, which in turn drives the treatment cartridge push plate 150 in the X⁺ direction and into the cartridge body 138. Consequently, the treatment substance, e.g., an antibiotic and/or sealant, retained within the treatment cartridge 14 is expelled/dispensed through the injection nozzle 42 into the streak canal of the teat.

Although the plunger assembly 18 and treatment cartridge have been exemplarily described above as being separate components that are removably engageable with each other, it is envisioned that the treatment cartridge 14 can be integrally formed with the plunger assembly 18 to provide a single component plunger and cartridge assembly.

The description herein is merely exemplary in nature and, thus, variations that do not depart from the gist of that which is described are intended to be within the scope of the teachings. Such variations are not to be regarded as a departure from the spirit and scope of the teachings.

What is claimed is:

1. An injection system for injecting a fluidic treatment substance into an orifice of an animal, said system comprising:
a treatment cartridge having a hollow body, the treatment cartridge structured and operable to retain a treatment substance within the hollow body; and
a plunger assembly structured and operable to removably retain the treatment cartridge and controllably dispense the treatment substance from the treatment cartridge.

2. The system of claim 1, wherein the treatment cartridge comprises one or more cartridge retention structures and the plunger assembly comprises a docking platform including one or more dock retention structures, wherein the one or more cartridge retention structures are structured and operable to mate with the one or more dock retention structures to removably retain the treatment cartridge within the docking platform.

3. The system of claim 1, wherein the treatment cartridge comprises an injection nozzle extending from the hollow body and a push plate slideably disposed within the hollow body, and the plunger assembly comprises a handle, a plunger rod slideably disposed within the handle, and a lever bar operatively connected to the plunger rod such that movement of the lever bar orthogonally toward the handle will advance the plunger rod within the handle so that the plunger rod will exert a force on the push plate of a treatment cartridge removably retained by the plunger assembly such that the push plate will force the treatment substance through the injection nozzle and dispense the treatment substance from the treatment cartridge.

4. The system of claim 3, wherein treatment cartridge further comprises a debris reservoir disposed within a surface of the push plate, the debris reservoir structured and operable to displace debris collected on a plunger cap of the plunger rod.

5. The system of claim 3, wherein the plunger assembly further comprises a biasing device structured and operable to bias movement of the lever bar orthogonally away from the handle.

6. The system of claim 3, wherein plunger assembly further comprises a hand buttress extending from the handle that is structured and operably to assist a user in maintaining a secure grip on the handle during use of the plunger assembly.

7. The system of claim 1 further comprising an injection site cleaning adaptor that is removably connectable to the treatment cartridge and is structured and operable to clean a site in which the treatment substance is to be injected.

8. The system of claim 7, wherein the cleaning adaptor comprises a pad basin and a cleaning pad pre-soaked with an antibacterial and/or an antimicrobial solution retained within the pad basin.

9. The system of claim 8, wherein the cleaning adaptor further comprises a removable seal disposed over the cleaning pad for preserving the integrity of the cleaning pad.

10. A treatment cartridge for use with a plunger assembly for injecting a fluidic treatment substance into an orifice of an animal, said cartridge comprising:
a hollow body;
an injection nozzle extending from the hollow body;
a push plate slideably disposed within the hollow body, the push plate structured and operable to move within hollow body, in response to a force applied by a plunger assembly, to force a treatment substance disposed within the hollow body through the nozzle to expel the treatment substance from the hollow body; and
one or more cartridge retention structures that are structured and operable to mate with the one or more dock retention structures of the plunger assembly to removably retain the treatment cartridge within the plunger assembly.

11. The cartridge of claim 10 further comprising a debris reservoir disposed within a surface of the push plate, the debris reservoir structured and operable to displace debris collected on a plunger cap of the plunger assembly.

12. The cartridge of claim 10, wherein the injection nozzle is structured and operable to be removably connectable to an injection site cleaning adaptor usable to clean a site in which the treatment substance is to be injected.

13. A method for injecting a fluidic treatment substance into an orifice of an animal, said method comprising:
removably retaining a treatment cartridge, comprising an injection nozzle extending from a hollow body and having a treatment substance disposed within the hollow body, within a plunger assembly comprising a handle, a plunger rod slideably disposed within the handle, and a lever bar operatively connected to the plunger rod; and
moving the lever bar orthogonally toward the handle to advance the plunger rod within the handle so that the plunger rod will interact with the treatment cartridge to expel the treatment substance through the injection nozzle, thereby dispensing the treatment substance from the treatment cartridge.

14. The method of claim 13, wherein removably retaining the treatment cartridge within the plunger assembly comprises removably mating one or more cartridge retention structures extending from the body of the treatment cartridge with one or more dock retention structures provided in a docking platform of the plunger assembly.

15. The method of claim 13, wherein moving the lever bar orthogonally toward the handle to expel the treatment substance through the injection nozzle comprises advancing the plunger rod within the handle so that a plunger cap of the plunger rod will exert a force on a treatment cartridge push plate slideably disposed within the hollow body of treatment cartridge such that the push plate will force the treatment substance through the injection nozzle, thereby dispensing the treatment substance from the treatment cartridge.

16. The method of claim 15, wherein moving the lever bar orthogonally toward the handle to expel the treatment substance through the injection nozzle further comprises overcoming a force provided by a plunger assembly biasing device that biases movement of the lever bar orthogonally away from the handle.

17. The method of claim 15, wherein moving the lever bar orthogonally toward the handle to expel the treatment substance through the injection nozzle further comprises utilizing hand buttress extending from the plunger assembly handle to obtain a secure grip on the handle during use of the plunger assembly.

18. The method of claim 13 further comprising cleaning a site in which the treatment substance is to be injected utilizing an injection site cleaning adaptor removably connectable to the treatment cartridge.

19. The method of claim 18, wherein cleaning the site in which the treatment substance is to be injected comprises cleansing the injection site utilizing a cleaning pad pre-soaked with an antibacterial and/or an antimicrobial solution that is retained within pad basin of the injection site cleaning adaptor.

20. The method of claim 19, wherein cleaning the site in which the treatment substance is to be injected further comprises removing a seal disposed over the cleaning pad for preserving the integrity of the cleaning pad.
